SEQUENCE LISTING (9 sequences)

SEQ ID NO: 1 - DNA, 18 bp, Artificial Sequence
  Description of Artificial Sequence Synthetic primer
tggggtgagggttgagtt

SEQ ID NO: 2 - DNA, 21 bp, Artificial Sequence
  Description of Artificial Sequence Synthetic primer
aatgttaacacaataggagcg

SEQ ID NO: 3 - DNA, 23 bp, Artificial Sequence
  Description of Artificial Sequence Synthetic primer
gctatttgctttgtccacctcta

SEQ ID NO: 4 - DNA, 24 bp, Artificial Sequence
  Description of Artificial Sequence Synthetic primer
aacctggaactgagaattgttcac

SEQ ID NO: 5 - DNA, 24 bp, Artificial Sequence
  Description of Artificial Sequence Synthetic primer
atcctttcaatctggaactgactg

SEQ ID NO: 6 - DNA, 24 bp, Artificial Sequence
  Description of Artificial Sequence Synthetic primer
gcattctaaaatcagggaaaatca

SEQ ID NO: 7 - DNA, 20 bp, Artificial Sequence
  Description of Artificial Sequence Synthetic primer
cttgaatgactgaagtttgg

SEQ ID NO: 8 - DNA, 16 bp, Artificial Sequence
  Description of Artificial Sequence Synthetic primer
cccacctgactcattt

SEQ ID NO: 9 - DNA, 20 bp, Artificial Sequence
  Description of Artificial Sequence Synthetic primer
ggtgtgtgtatgtacaactt

(19) United States
(12) Patent Application Publication
(10) Pub. No.: US 2010/0144776 A1
(43) Pub. Date: Jun. 10, 2010

(54) USE OF SNPS FOR THE DIAGNOSIS OF A PAIN PROTECTIVE HAPLOTYPE IN THE GTP CYCLOHYDROLASE 1 GENE (GCH1)

(75) Inventors: Irmgard Tegeder, Frankfurt am Main (DE); Joern Loetsch, Frankfurt am Main (DE)

Correspondence Address: DAVID S. RESNICK, NIXON PEABODY LLP, 100 SUMMER STREET, BOSTON, MA, 02110-2131, US

(73) Assignee: JOHANN WOLFGANG GOETHE-KUNIVERSITAET FRANKFURT AM MAIN, Frankfurt am Main (DE)

(21) Appl. No.: 12/440,507

(22) PCT Filed: Sep. 7, 2007
(86) PCT No.: PCT/EP2007/008039

§ 371 Date: Jan. 20, 2010

Related U.S. Application Data
(60) Provisional application No. 60/843,078, filed on Sep. 8, 2006.

(51) Int. Cl. A61K 31/485; C12Q 1/68; A61P 25/04

(52) U.S. Cl. 514/282; 435/6

(57) ABSTRACT

The present invention relates to an in vitro method for diagnosing a genetic predisposition or susceptibility for pain in a mammal which comprises detecting of at least one particular single nucleotide polymorphism (SNP) in a sample obtained from said mammal in the genomic locus-derived nucleic acid or fragment thereof of the locus GCH1.

The present invention relates to an in vitro method for diagnosing a genetic predisposition or susceptibility for pain in a mammal which comprises detecting of at least one particular single nucleotide polymorphism (SNP) in a sample obtained from said mammal in the genomic locus-derived nucleic acid or fragment thereof of the locus GCH1.

### BACKGROUND OF THE INVENTION

Genetic factors explain an increasing fraction of the inter-individual variability in the development and treatment of pain. Polymorphic genes mediate the individual susceptibility to develop pain in pathological conditions, the individual response to experimental painful stimuli, and the individual response to analgesic pharmacological treatment.

Kealey C et al. (Kealey C, Roche S, Claffey E, McKeon P. Linkage and candidate gene analysis of 14q22-24 in bipolar disorder: support for GCHI as a novel susceptibility gene. Am J Med Genet B Neuropsychiatr Genet. 2005 Jul. 5; 136(1):75-80.) describe a linkage implicating 14q22-24 in bipolar disorder (BPD). A web-based candidate gene search of 14q22-24 resulted in the selection of GTP cyclohydrolase I (GCHI), located 200 kb 3′ of D14S281, as the best plausible candidate gene for involvement in BPD. An association study between BPD and a novel single nucleotide polymorphism (SNP) in GCHI (G to A at position −959 bp, upstream of the ATG codon), is also presented.

Ichinose H et al. (Ichinose H, Ohye T, Matsuda Y, Hori T, Blau N, Burlina A, Rouse B, Matalon R, Fujita K, Nagatsu T. Characterization of mouse and human GTP cyclohydrolase I genes. Mutations in patients with GTP cyclohydrolase I deficiency. J Biol. Chem. 1995 Apr. 28; 270(17):10062-71.) describe a characterization of the GTP cyclohydrolase I gene and multiple species of mRNA, as well as a structural analysis.

WO 2005-048926 describes methods and compositions for preventing, reducing, or treating a traumatic, metabolic or toxic peripheral nerve lesion or pain including, for example, neuropathic pain, inflammatory and nociceptive pain by administering to a mammal in need thereof a compound that reduces the expression or activity of BH4. This reduction may be achieved by reducing the enzyme activity of any of the BH4 synthetic enzymes, such as GTP cyclohydrolase (GTPCH), sepiapterin reductase (SPR), or dihydropteridine reductase (DHPR); by antagonizing the cofactor function of BH4 on BH4-dependent enzymes; or by blocking BH4 binding to membrane bound receptors. The application also provides methods for diagnosing pain or a peripheral nerve lesion in a mammal by measuring the levels of BH4 or its metabolites in biological sample.

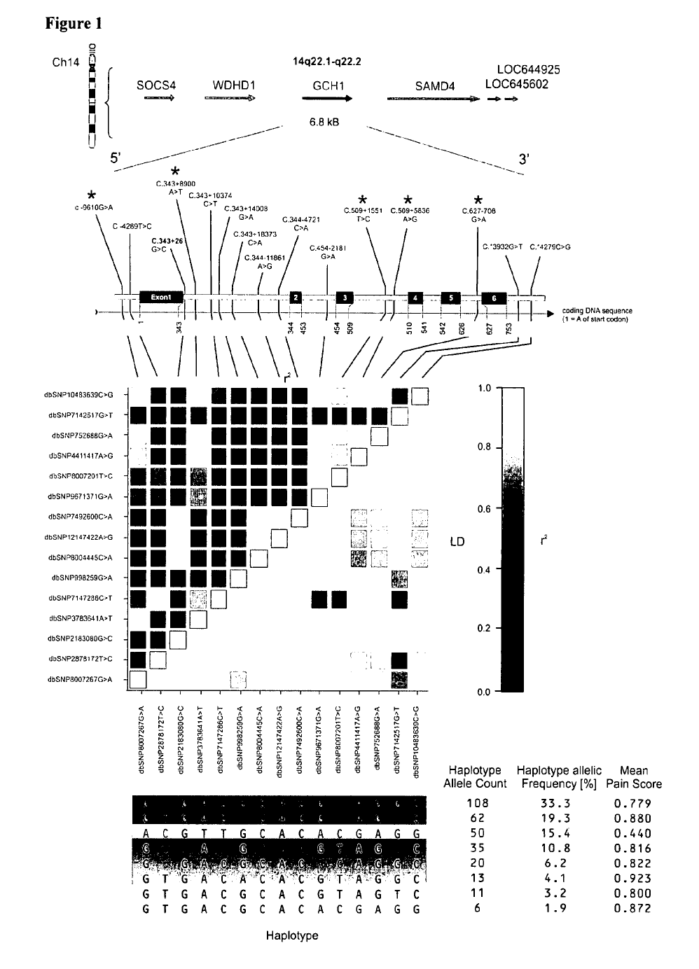

Recently, a haplotype in the GTP cyclohydrolase 1 gene ([1]; GCH1; FIG. 1) has been associated with decreased persistent radicular pain after surgical diskectomy and reduced experimental pain in volunteers (2). The enzyme is rate-limiting for the synthesis of tetrahydrobiopterin, an essential cofactor for enzymes involved in catecholamine, serotonin and nitric oxide synthesis. GTP cyclohydrolase 1 is up-regulated in primary sensory neurons following peripheral nerve injury. Its inhibition reduces nociceptive responses in various models of neuropathic and inflammatory pain and tetrahydrobiopterin produces pain in naïve animals and further increases persistent pain (2). As tetrahydrobiopterin is an essential cofactor for nitric oxide and serotonin synthesis, both previously implicated in pain pathways, modulation of these mediators may contribute to the pain-producing effects of tetrahydrobiopterin. Without wanting to be bound by theory, the functional consequence of the pain protecting haplotype is a reduction of stimulated tetrahydrobiopterin synthesis due to reduced upregulation of mRNA and protein of GCH1.

In addition to the above, reliable markers for a genetic analysis of a pain protective phenotype are needed in order to expand and improve the reliability of said methods in order to fully exploit the diagnostic potential for a particular patient and/or subject.

Recently, a haplotype composed of 15 positions of the GCH1 gene was been identified as being associated with pain protection (2). The diagnosis based on the full genetic information would require a considerable laboratory effort including both the identification of 15 DNA positions and haplotype assignment. A screening assay requiring substantially less laboratory diagnostic effort is thus desirable in order to ease the application of GCH1 genetics in clinical research and treatment of pain, and in particular neuropathic pain.

The object of the present invention, in a first preferred aspect thereof, is achieved by a method for diagnosing a genetic predisposition or susceptibility for developing acute and/or chronic pain in a mammal, comprising detecting in a sample obtained from said mammal at least one single nucleotide polymorphism (SNP) in a nucleic acid or fragment thereof derived from the genomic locus of the gene GCH1, wherein said at least one SNP is selected from the group consisting of the SNPs rs8007267 G>A, rs3783641 A>T, rs8007201 T>G, rs4411417 A>G, rs752688 G>A, and rs10483639 C>G, preferably from the group consisting of the SNPs rs8007267 G>A, rs3783641 A>T, and rs10483639 C>G. The method can be an in vitro, an in vivo, or an in-situ method. Preferred are SNPs rs8007267 G>A and rs3783641 A>T, more preferably together with rs10483639 C>G.

Using 290 DNA samples genotyped for all 15 positions, the present inventors show that a diagnosis of the pain protective haplotype is surprisingly possible in 100% of the cases by using only three GCH1 DNA positions. Moreover, the inventors could show that the 100% correct haplotype assignment does not require in-silico haplotyping, but can be obtained on a “simple” SNP basis. The intention of a screening assay to substantially ease the genetic diagnosis of the pain protective haplotype has thus been achieved by a reduction of the GCH1 SNPs from 15 to only three. In addition, the present invention provides a rapid and reliable detection of said three SNPs as has been achieved by the development of pyrosequencing assays, as will be explained in more detail below.

The detected allelic frequencies of the three SNPs corresponded for both, the 290 pain patients from (2), and the 629 randomly selected healthy volunteers who's DNA served for pyrosequencing assay design, to the allelic frequencies known for Caucasian samples (NCBI SNP database at http://www.ncbi.nlm.nih.gov/SNP/) There are a few interethic differences in the allelic frequency of the three SNPs.

The dbSNP rs8007267 has a similar frequency among Caucasians, Chinese, Japanese (14%-18%) but is double as frequent in African Americans (34%, source: Applied Biosystems website, can be reached via the link on the dbSNP website of this SNP). In contrast, dbSNP rs10483639 is rarer in Caucasians (23%) than in the other above-mentioned ethnicities (35%-41%). Since a haplotype cannot be more frequent than the rarest allele of which it is composed, this pattern of SNP frequencies opens the possibility that the pain protective GCH1 haplotype is more frequent among African Americans than presently found in Caucasians. In contrast, the SNP frequency pattern does not indicate a difference in the haplotype frequency for the other ethnicities as compared to Caucasians. Evidently, the complexity of the haplotype requires direct assessment of its frequency in other ethnicities, and the present speculations cannot provide more than a sensitization toward possible interethnic difference in the pain protective GCH1 haplotype's allelic frequency, which has to be addressed in future evaluations of its clinical role.

The GCH1 haplotype identified according to the present invention that is linked to protection against the development of pain, and in particular neuropathic pain (in the following also “pain protective”), is the third described GCH1 phenotype based on GCH1 genetic variants. Diseases associated with GCH1 mutations are DOPA responsive hereditary progressive dystonia (11), and atypical phenylketonuria (12), caused by GCH1 mutations in coding regions or by deletion of a large part of the gene including exon deletions (11, 13, 14). These rare GCH1 variants cause deleterious defects in dopamine synthesis or phenylalanine metabolism due to tetrahydrobiopterin cofactor deficiency. The inventive pain protective GCH1 haplotype is not associated with any neurologic dysfunctions or other overt pathology.

The SNPs forming the pain protective GCH1 haplotype are all localized in non-coding regions of the gene. Without wanting to be bound by theory, their localization in the promoter, introns and the 3′ downstream region suggests that they may cause decreased GCH1 transcription or RNA stability, which is in agreement with the observed lower GCH1 mRNA expression in forskolin stimulated monocytes from carriers of the pain protective haplotype as compared to controls (2).

The screening assay forming the basis for the present invention was designed in order to reliably detect the complete haplotype associated with pain protection (2). The selection of the three SNPs was based on identification of single alleles or combinations of alleles of the 15 GCH1 DNA positions that were unique for the pain protecting haplotype. This is independent of the relative functional importance of particular SNPs within the complete 15-position haplotype for providing GCH1 genotype/phenotype associations. The two GCH1 SNPs with the highest level of statistical significance of their association with a low pain score are included in the present screening assay, namely dbSNP rs8007267G>A and dbSNP rs3783641A>T. Most preferred is a method according to the present invention, wherein the inventive diagnosis identifies individuals that are protected from said acute and/or chronic pain, and in particular neuropathic pain.

Although the method of the invention can be performed in mammals in general, it is preferred to perform the method with human samples. Such samples may be, e.g. blood, urine, semen, hair or any other tissue containing at least the nucleic acid to be analyzed.

The nucleic acid that is part of the method according to the present invention can be DNA, genomic DNA, RNA, cDNA, hnRNA and/or mRNA. The detection can be accomplished by sequencing, mini-sequencing, hybridisation, restriction fragment analysis, oligonucleotide ligation assay, or allele specific PCR.

Applicable diagnostic techniques include, but are not limited to, DNA sequencing including mini-sequencing, primer extension, hybridization with allele-specific oligonucleotides (ASO), oligonucleotide ligation assays (OLA), PCR using allele-specific primers (ARMS), dot blot analysis, flap probe cleavage approaches, restriction fragment length polymorphism (RFLP), kinetic PCR, and PCR-SSCP, fluorescent in situ hybridisation (FISH), pulsed field gel electrophoresis (PFGE) analysis, Southern blot analysis, single stranded conformation analysis (SSCA), denaturing gradient gel electrophoresis (DGGE), temperature gradient gel electrophoresis (TGGE), denaturing HPLC (DHPLC), and RNAse protection assays, all of which are known to the person skilled in the art and discussed in detail further below.

The presence of a polymorphism/mutation can be determined by extracting DNA from any tissue of the body. For example, blood can be drawn and DNA extracted from blood cells and analyzed. Moreover, prenatal diagnosis of the condition will be possible by testing fetal cells, placental cells or amniotic cells for mutations in the gene. There are several methods that allow the detection of specific alleles, and some of these methods are discussed here:

For a known polymorphism, direct determination of the respective genotype is usually the method of choice. State of the art approaches for industrial high-throughput genotyping today rely on one of four different mechanisms: allele-specific primer extension, allele-specific hybridization, allele-specific oligonucleotide ligation and allele-specific cleavage of a flap probe (Kwok, Pharmacogenomics 1, 95 (2000)). Sequencing or mini-sequencing protocols are part of the primer extension methods, e.g. genomic DNA sequencing, either manual or by automated means. Minisequencing (primer extension) technology is based on determining the sequence at a specific base by allowing the elongation of a primer by one base directly at the variant site (Landegren et al., Genome Res. 8: 769-76 (1998)). Short sequence reactions coupled with an alternative detection method are the nature of real time pyrophosphate sequencing (Nyren et al., Science 281:363 (1998)).

Allele-specific hybridization protocols rely on probes detecting one or several of the alleles present at the SNP positions. Several techniques were developed for detection of an hybridization event. In the 5′ nuclease assay and in the molecular beacon assay the hybridization probes are fluorescently labeled and probe binding is detected via changes in the behavior of the fluorescent label (Livak, Genet. Anal. 14, 143 (1999); Tyagi et al., Nat. Biotechnol. 16, 49 (1998)). Hybridization events may occur in liquid phase or with either the probe or the target bound to a solid surface.

Hybridization is thus also used when arrays (microchips) are used for genotyping purposes. This technique of nucleic acid analysis is also applicable to the present invention. An array typically consists of thousands of distinct nucleotide probes which are built up in an array on a silicon chip. Nucleic acid to be analyzed is fluorescently labeled, and hybridized to the probes on the chip. This method is one of parallel processing of thousands of probes at once and can tremendously accelerate the analysis. In several publications the use of this method is described (Hacia et al., Nature Genetics 14, 441 (1996); Shoemaker et al., Nature Genetics 14, 450 (1996); Chee et al., Science 274, 610 (1996); DeRisi et al., Nature Genetics 14, 457 (1996), Fan et al., Genome Res, 10, 853 (2000)).

Allele-specific oligonucleotide ligation assays have a high specificity. Oligonucleotides differing in the allele-specific base at the 5′- or 3′-end are only processed in a ligation reaction if they are perfectly bound to the template at the respective oligonucleotide end. This method has been coupled with fluorescence resonance energy transfer (FRET) labeling to create a homogeneous assay system (Chen et al. Genome Res. 8, 549 (1998)). Allele-specific cleavage of a flap probe use the property of recently discovered flap endonucleases (cleavases) to cleave structures created by two overlapping oligonucleotides. In this approach two overlapping oligonucleotides are bound to the polymorphic site. That oligo which has had a perfect match to the target sequence is then detected by the cleavage reaction (Lyamichev et al., Nat. Biotechnol. 17:292 (1999)).

Other methods which detect specific base variations usually allow only a lower throughput, such as the allele-specific oligonucleotide (ASO) hybridization. For allele-specific PCR, primers are used which hybridize at their 3′ ends to one of the particular GCH1 base variations according to the invention. Only for alleles which are present, a respective PCR product is generated (Ruano and Kidd, Nucleic Acids Res 17, 8392 (1989)). A specificity increasing modification of allele-specific PCR is the Amplification Refractory Mutation System, as disclosed in European Patent Application Publication No. 0332435 and in Newton et al., Nucleic Acids Res 17, 2503 (1989). If the variations lead to changes in the specific recognition sites of nucleic acid processing, enzymes methodologies such as restriction fragment length polymorphism (RFLP) probes or PCR-RFLP methods may also be used to detect these variations.

Other approaches can only detect that changes with respect to a reference sequence are present in a nucleic acid. Many of these methods are based on the formation of mismatches when both SNP variants are present in the same sample. A currently very popular method is the use of denaturing high performance liquid chromatography to separate heteroduplex from homoduplex molecules (DHPLC; Oefner, P. J. et al. Am J Hum Genet. 57 (Suppl.), A266 (1995)). Another method is denaturing gradient gel electrophoresis (DGGE) (Wartell et al., Nucleic Acids Res 18, 2699, (1990); Sheffield et al., Proc Natl Acad Sci USA 86, 232 (1989)). By using DGGE, variations in the DNA can be detected by differential migration rates of allelic variants in a denaturing gradient gel. A variation is the clamped denaturing gel electrophoresis (CDGE; Sheffield et al., Am J Hum Genet. 49, 699 (1991)), heteroduplex analysis (HA; White et al., Genomics 4, 560 (1992)) and chemical mismatch cleavage (CMC; Grompe et al. Proc Natl Acad Sci USA 86, 5888 (1989)). The use of proteins which recognize nucleotide mismatches, such as the E. coli mutS protein may help in detecting mismatched DNA molecules (Modrich, Ann. Rev. Genetics, 25, 229 (1991)). In the mutS assay, the protein binds only to sequences that contain a nucleotide mismatch in a heteroduplex between mutant and wild-type sequences. RNase protection assays are another option (Finkelstein et al., Genomics 7, 167 (1990)). The RNAse protection assay involves cleavage of the mutant fragment into two or more smaller fragments. Another way is to make use of the single-stranded conformation polymorphism assay (SSCP; Orita et al., Proc Natl Acad Sci USA 86, 2766 (1989)). Variations in the DNA sequence of the gene from the reference sequences will be detected due to a shifted mobility of the corresponding DNA-fragments in SSCP gels. SSCP detects bands which migrate differently because the variation causes a difference in single strand, intra-molecular base pairing.

Indirect methods as described above for the detection of sequence variations would be particularly useful for screening relatives for the presence of a sequence variation found previously in an affected family member. Other approaches for detecting small sequence variations as known for those skilled in the art can be used.

Detection of polymorphisms/point mutations may be accomplished by amplification, for instance by PCR, from genomic or cDNA and sequencing of the amplified nucleic acid or by molecular cloning of the GCH1 allele and sequencing the allele using techniques well known in the art.

In a preferred embodiment of the present invention, at least one of the nucleotide alterations of the gene GCH1 is detected in a sample by hybridizing a gene probe which specifically hybridizes to the alternative forms of the polymorphism/variant nucleic acids containing at least one of said alterations of the gene from said mammalian sample and detecting the presence of a hybridization product, wherein the presence of said product indicates the presence of said base configuration in said sample.

Herein, the gene probes are e.g. oligomeric DNA sequences of 15 to 50 bases which are synthesized with at least one variant base, preferentially both variant bases and hybridized individually under stringent conditions allowing single base variant discrimination. Alternatively, under less stringent conditions, a set of gene probes of 15 to 50 bases in length representing all four potential bases at the polymorphic position of the analyzed DNA strand can be used for typing by hybridization. In this case, results from all hybridization experiments with differing degrees of base complementarity need to be analyzed by an algorithm to predict the final nucleotide configuration at the variant site.

Preferred is a method according to the present invention, wherein a) at least one SNP is detected by amplifying all or part of a GCH1 nucleic acid in said sample using a set of specific primers to produce amplified GCH1 nucleic acids, b) sequencing, e.g. mini-sequencing, the amplified nucleic acids and c) detecting the presence of the at least one SNP and thereby the presence of said nucleotide alterations in said sample. Preferably, the primer can further contain a detectable label, e.g. a radionuclide, fluorophore, peptide, enzyme, antigen, antibody, vitamin or steroid.

Preferably, a combination of the SNP alleles of the GCH1 is detected, consisting of one, two, or preferably all three SNPs alleles per locus. Also preferred is a method according to the present invention, wherein a combination together with other statistically significant SNPs in the gene GCH1 is analyzed, such as, for example, an SNP chosen from the 12 other SNPs in GCH1, as disclosed in (2) and/or according to the following table.

TABLE 1
Locations and allelic frequencies of 15 GCH1 SNPs

| | | | Allelic | | | | | | | |
|---|---|---|---|---|---|---|---|---|---|---|
| | | | frequency | | | | | | | |
| | Location | Allelic | of | | | | | | | |
| | relative to | variation | uncommon | Number of | | | Mean pain z-score | | | Regression |
| | coding | common > | allele | patients^{a} | | | for “leg pain” | | | analysis |
| dbSNP ID | region | uncommon | (%) | 0/0 | 0/1 | 1/1 | 0/0 | 0/1 | 1/1 | P-value |
| rs8007267 | C.−9610 | G > A | 17.50 | 108 | 48 | 4 | 0.81 | 0.48 | 0.06 | 0.0128 |
| rs2878172 | C.−4289 | T > C | 37.42 | 64 | 71 | 24 | 0.92 | 0.57 | 0.69 | 0.1262 |
| rs2183080 | C.343 + 26 | G > C | 11.18 | 129 | 28 | 4 | 0.77 | 0.63 | 1.57 | 0.6424 |
| rs3783641 | C.343 + 8900 | A > T | 17.41 | 108 | 45 | 5 | 0.82 | 0.51 | 0.15 | 0.0212 |
| rs7147286 | C.343 + 10374 | C > T | 29.69 | 81 | 63 | 16 | 0.89 | 0.49 | 0.82 | 0.1256 |
| rs998259 | C.343 + 14008 | G > A | 25.63 | 89 | 60 | 11 | 0.67 | 0.79 | 0.95 | 0.2746 |
| rs8004445 | C.343 + 18373 | C > A | 10.94 | 129 | 27 | 4 | 0.78 | 0.63 | 1.58 | 0.6559 |
| rs12147422 | C.344 − | A > G | 11.25 | 128 | 28 | 4 | 0.76 | 0.66 | 1.56 | 0.5322 |
| | 11861 | | | | | | | | | |
| rs7492600 | C.344 − 4721 | C > A | 11.25 | 128 | 28 | 4 | 0.76 | 0.67 | 1.57 | 0.5250 |
| rs9671371 | C.454 − 2181 | G > A | 25.63 | 87 | 61 | 10 | 0.81 | 0.59 | 0.32 | 0.0537 |
| rs8007201 | C.509 + 1551 | T > C | 25.63 | 90 | 58 | 12 | 0.81 | 0.61 | 0.21 | 0.0300 |
| rs4411417 | C.509 + 5836 | A > G | 18.13 | 109 | 44 | 7 | 0.81 | 0.54 | 0.18 | 0.0279 |
| rs752688 | C.627 − 708 | G > A | 18.01 | 110 | 44 | 7 | 0.80 | 0.54 | 0.18 | 0.0289 |
| rs7142517 | C.*3932 | G > T | 35.76 | 67 | 69 | 22 | 0.60 | 0.76 | 0.93 | 0.1360 |
| rs10483639 | C.*4279 | C > G | 18.13 | 109 | 44 | 7 | 0.79 | 0.58 | 0.19 | 0.0516 |

Also preferred is a method according to the present invention, wherein a combination together with other statistically significant SNPs in a gene selected from the group of KCNS1, OPMR1 (e.g. SNP at 118 A>G), COMT, and PGHS2 (e.g. SNP at −765 G>A) is analyzed. Preferably, the detection allows for a detection of the pain protective haplotype with a sensitivity >0.80 (preferably of about >0.90) and a specificity of about 0.70 or more, preferably about 0.9 or more.

In the context of the present invention, the term “sensitivity” (commonly also termed true positive rate) of a statistic test or another classification indicates the probability, to recognize a positive result. Thus, it gives the proportion of the results that have been correctly identified as positive (true positive) of a total of truly existing positive results. For example, the sensitivity in a medical examination/diagnostic method for determining a disease will indicate the proportion of diseased patients that have been correctly identified as having the disease.

In the context of the present invention, the term “specificity” (commonly also termed true negative rate) of a statistic test or another classification indicates the probability, to recognize a negative result. Thus, it gives the proportion of the results that have been correctly identified as negative (true negative) of a total of truly existing negative results. For example, the specificity in a medical examination/diagnostic method for determining a disease will indicate the proportion of diseased patients that have been correctly identified as not having the disease.

Another aspect of the present invention is directed to a method according to the invention, further comprising an analysis of biopterin in whole blood and/or isolated leukocytes with and without a cellular stimulation, such as with forskolin, LPS, or the like. An exemplary method for an analysis of biopterin is described in (2). It is expected that such a combined analysis with biopterin will increase a functional predictability of the method according to the present invention, and is optimally a synergistic increase.

Another aspect of the present invention is directed to a method for producing an effective analgesic composition, comprising a) performing a method according to the present invention as above, and b) determining the effective dosage of an analgesic substance for said mammal based, at least in part, on the result as obtained in step a), and c) admixing said dosage with a pharmaceutically acceptable carrier and/or diluent.

While, in general, a treatment of pain and respective analgesic compositions are well known by the person of skill, the present invention, in this aspect thereof, provides a basis for a “personalized” treatment of pain of an individual patient and/or a group of patients that may react differently to said treatment, compared to other groups of patients. The present method is particularly advantageous in that it helps to reduce unwanted side effects of a medication (for example, by overdosing), helps to reduce the dosing of toxic or addictive substances (such as, for example, opioids) and can thus help to save costs by avoiding unnecessary treatments and expensive medications that are ineffective. Analgesic substances as such and how analgesic compositions are formulated is well known by the person of skill and extensively described in the respective literature. The present method is particularly advantageous in that it helps to assess the risk for chronic pain, e.g. in patients with viral infections (e.g. zoster, HIV) or potentially neurotoxic treatment (chemotherapy, radiation or other drugs) or surgery that involves nerve damage (e.g. herniotomy, mastectomy).

Consequently, another aspect of the present invention is directed to an improved method of treating pain in a mammal, comprising a) a method according to the present invention as above, and b) providing an analgesic substance to said mammal based, at least in part, on the result as obtained in step a). Preferred is a method according to the present invention, wherein the effective analgesic composition according to the present invention as above is administered.

Finally, the invention is also directed to a diagnostic kit and/or a research kit that comprises at least one probe and/or set of primers for detecting at least one of the SNPs of the gene GCH1, selected from the group consisting of the SNPs rs8007267 G>A, rs3783641 A>T, rs8007201 T>G, rs4411417 A>G, rs752688 G>A, and rs10483639 C>G, preferably from the group consisting of the SNPs rs8007267 G>A, rs3783641 A>T, and rs10483639 C>G. The kit can contain other compounds such as enzymes, buffers, and/or dyes for performing the method(s) of the present invention. In another example, the kit according to the invention is suitable to perform a chip-based analysis in at least one SNP according to the invention. The kit can also include instructions for performing the SNP-analysis and/or the software for a statistical analysis as described herein.

“Predisposed” or “susceptible to pain” in the context of the present invention shall mean that the individual under examination experiences a longer, more frequent or more intensive sensation of pain, compared to individuals that have a respectively shorter, less frequent or less intense pain sensations.

According to another aspect of the present invention, the inventive method allows for the identification of individuals that are protected from pain and pain involving conditions, i.e. are less likely to suffer from said pain. Preferably, said genetic predisposition or susceptibility involves pain caused or contributed to by nerve injury (e.g. traumatic, ischemic, toxic, metabolic, infectious, immune-mediated, constrictive, degenerative, etc.), inflammation (e.g. infectious, immune-mediated), ischemia, or tumor growth.

In summary, the inventors have developed a potentially high-throughput automatable screening assay for a pain protective GCH1 haplotype consisting of three SNPs. By informational analysis it is show that the number of DNA positions to be genotyped can be reduced to these three, still allowing reliable diagnosis of the haplotype. This substantially decreases the laboratory effort for its diagnosis and thus facilitates further investigations of the clinical importance of the pain protective GCH1 haplotype.

For the purposes of the present invention, all references as cited herein are incorporated by reference in their entireties. The present invention shall now be further described in the following examples with respect to the accompanying drawings without being limited thereto, wherein

FIG. 1 shows an overview of locations of GCH1 single nucleotide polymorphisms (Ensemble database v.38—April 2006), those significantly associated with low pain scores are coded in light grey (*P<0.05; pain scores for each SNP in Table 1). Genotype-phenotype associations of eight haplotypes with frequency >1% and accounting for 94% of chromosomes studied, were analyzed. Letters in each haplotype are alleles for the 15 GCH1 SNPs. Pain scores for each haplotype are the mean z-score for “leg pain” calculated from four questions assessing frequency of pain at rest, after walking, and their improvement after surgery adjusted for covariates. Lower scores correspond to less pain. The highlighted haplotype (white) was associated with lower “leg pain” scores than the seven other haplotypes; P 0.009. Prior to the analyses, the inventors specified a single primary endpoint, persistent leg pain over the first postoperative year after diskectomy, as a reflection of ongoing neuropathic pain. Leg pain was assessed before surgery and at 3, 6 and 12 months after surgery by four items: Frequencies in the past week of “leg pain”, and of “leg pain after walking”, were rated as never (0 points), very rarely (1), a few times (2), about ½ the time (3), usually (4), almost always (5), and always (6). Improvements in “leg pain” or in “leg pain after walking” since surgery were rated as pain completely gone (0), much better (1), better (2), a little better (3), about the same (4), a little worse (5), and much worse (6). For each variable in each patient, the inventors calculated an area-under-the-curve score for the first year and standardized the patients' AUC scores for each variable to z-scores which have a mean equal to 0 and standard deviation equal to 1. The primary pain outcome variable was the mean of these four z-scores. Genotype-phenotype associations for each SNP were sought by regression analysis using the equation: Individual pain score=(R1*number of uncommon alleles)+(R2*covariates)+error. (R1, R2=regression coefficients). The covariates were a number of demographic, psychological and environmental factors, including sex, age, workman's compensation status, delay in surgery after initial enrollment, and Short-Form 36 (SF-36) general health scale. Stepwise regression was applied to assess the association between pain scores and diplolotypes by modeling pain scores as a function of all haplotypes generated by the 15 GCH1 SNPs and of relevant covariates. Only haplotypes with frequencies >1% were included in the model and were used as independent variables. If a haplotype was associated with a pain score that differed significantly from the average pain score (P<0.05), phenotype-diplotype association analysis was performed by regression analysis using a similar model as described above for individual SNPs.

### EXAMPLES

The sequences of the GCH1 gene on chromosome 14q22.1-q22.2 were obtained from databases Ensembl Gene ID ENSG00000131979 at http://www.ensembl.org/Homo_sapiens/geneview?db=core; gene=ENSG00000131979. SNPs are named according to the NCBI SNP database http://www.ncbi.nlm.nih.gov/SNP/ (dbSNP, followed by the accession number).

### SNP Selection for GCH1 Genetic Screening

The selection of the GCH1 SNPs for the screening assays was based on (i) the distinctive property for the pain GCH1 haplotype, (ii) the statistical significance (2) for the primary outcome of leg pain over the first 12 months following lumbar diskectomy, and (iii) the sensitivity and specificity of the resulting screening assay to detect the pain protective haplotype, calculated as sensitivity=correctly positive/(correctly positive+false negative), and specificity=correctly negative/(correctly negative+false positive).

The original (2) GCH1 genetic data set consisted of 290 DNA samples from patients after surgical diskectomy with the 15 GCH1 single nucleotide polymorphisms (SNP), screened for by means of the 5′-exonuclease method (6). GCH1 haplotypes had been identified by means of in-silico haplotyping with PHASE (http://www.stat.washington.edu/stephens/software.hml) [7, 8]. The pain protective haplotype #3 (2) is distinct among the ten most frequent haplotypes (amounting to a total of 94.4%) by the adenine at SNP 1 (dbSNP rs8007267 G>A=GHC1 promoter SNP-9610 G>A) together with the thymine at DNA SNP 4 (dbSNP rs3783641 A>T, the, 8900^{th} nucleotide of intron 1). Correct assignment of the 290 individuals to the pain protective haplotype on the basis of these two DNA positions was assessed by means of in-silico haplotyping with PHASE (7, 8). Since this resulted in a specificity below 1, a third SNP, dbSNP rs10483639 C>G, was included into the screening assay, which provided the desired test sensitivity and specificity of 1.

### Pyrosequencing Screening Assays

### DNA Extraction

The DNA samples for screening assay development were obtained from 629 healthy unrelated subjects (age 27.1+/−5.5 years) of Caucasian ethnicity who had consented into genotyping. They were recruited via flyers at the Frankfurt University Hospital. The procedure had been approved by the Medical Faculty Ethics Committee of the Johann Wolfgang Goethe University of Frankfurt. Blood samples were dram into NH4-heparin tubes. Genomic DNA was extracted from 200 μl blood using the “blood and body fluid spin protocol”, provided in the EZ1 DNA Blood 200 μl Kit on a BioRobot EZ1 Workstation (Qiagen. Hilden, Germany).

### Assay Design

In pyrosequencing (9, 10), a short oligonucleotide (sequencing primer) binds at the single strand DNA close to the mutation and is elongated by dispensing deoxynucleotide triphosphates (dNTP). If the dispensed dNTP matches the next nucleotide of the DNA sequence, it is incorporated into the oligonucleotide and pyrophosphate (PPi) is released (DNAn+dNTP+DNAn+1+PPi). The PPi is used together with adenosine 5-phosphosulfate (APS) as a substrate for ATP sulfurylase. The resulting ATP triggers the luciferase catalyzed conversion of luciferin to oxiluciferin, emitting light of intensity proportional to the number of added nucleotides. It is visualized as a peak in the so-called programs, whereas no peak is observed in case of non-incorporation.

The primers necessary for PCR amplification of GCH1 gene segments of interest (portions of promoter/5′UTR, of intron 1, and of the 3′UTR) and the sequencing primers were designed using the pyrosequencing Assay Design Software (version 1.0.6; Biotage AB, Uppsala, Sweden). PCR primers for

SNP dbSNP rs8007267 G>A were:

(SEQ ID No. 1)

forward: 5′-TGGGGTGAGGGTTGAGTT-3′,
and

(SEQ ID No. 2)

reverse: 5′-biotin-AATGTTAACACAATAGGAGCG-3′,

for dbSNP rs3783641 A>T were;

(SEQ ID No. 3)

forward: 5′-GCTATTTGCTTTGTCCACCTCTA-3′,
and

(SEQ ID No. 4)

reverse: 5-biotin-AACCTGGAACTGAGAATTGTTCAC-3′

and

for dbSNP rs10483639 C>G were

(SEQ ID No. 5)

forward: 5′-ATCCTTTCAATCTGGAACTGACTG-3′,
and

(SEQ ID No. 6)

reverse: 5′-biotin-GCATTCTAAAATCAGGGAAAATCA-3′.

The sequencing primers were

| | |
|---|---|
| 5′-CTTGAATGACTGAAGTTTGG-3′, | (SEQ ID No. 7) |

for dbSNP rs8007267 G>A,

| | |
|---|---|
| 5′-CCCACCTGACTCATTT-3′, | (SEQ ID No. 8) |

for dbSNP rs3783641 A>T, and

| | |
|---|---|
| 5′-GGTGTGTGTATGTACAACTT-3′, | (SEQ ID No. 9) |

for dbSNP rs10483639 C>G.

The specificity of the primers for the GCH1 gene was verified by alignment (www.ncbi.nlm.nih.gov/BLAST). In addition, the software defined the dNTP dispensation orders for detection of the three SNPs. Likewise, all primer sequences for the other SNPs as disclosed herein can be designed using the same methodology.

### PCR Amplification

Polymerase chain reactions were performed in a total volume of 50 μl. After PCR amplification, several samples were controlled on ethidium bromide-stained agarose gels where the specific product bands were seen (321 bp PCR product for the SNP dbSNP rs8007267 G>A, 216 bp for dbSNP rs3783641 A>T, and 161 bp for dbSNP rs10483639 C>G).

A volume of each 25 μl PCR-template (biotinylated and non-biotinylated single strands) was pipetted into one well and immobilized by incubation (shaker 800 min⁻¹, 5 min. room temperature) with a mixture of 3 μl streptavidin-coated sepharose beads (Amersham Pharmacia Biotech, Uppsala, Sweden), 37 μl binding buffer and 15 W HPLC-purified water. Specific complexes are made of streptavidin-coated sepharose beads and biotinylated single strands.

The separation of these complexes from the non-biotinylated single strands was performed on a Vacuum Prep Worktable (Biotage AB, Uppsala, Sweden). After removal of all liquid by vacuum, the specific complexes were captured, transferred into 70% ethanol for 5 S, denaturized in 0.2 mol/l NaOH for 5 s, and washed with Tris buffer for 5 s. Then, the complexes were transferred to a PSQ 96 Plate Low (Biotage AB, Uppsala, Sweden), pre-filled with 0.15 μl of 10 μmol/l sequencing primer and 43.5 μl annealing buffer (20 mmol/l Tris, 2 mmol/l Mg acetate tetrahydrate at pH 7.6). Subsequently, the plate was heated at 80° C. for 2 min in a PSQ 96 Sample Prep Thermoplate Low (Biotage AB, Uppsala, Sweden), and cooled down to room temperature.

### Pyrosequencing Analysis

Sequencing took place at a PSQ 96MA (Biotage AB, Uppsala, Sweden) using enzymes, substrate and nucleotides as provided (Pyro Gold Reagents Kit for SNP Genotyping and Mutation Analysis, Biotage AB, Uppsala, Sweden). All buffers were prepared according to the recommended operating procedure of Sepharose Bead Sample Prep Buffer preparation (Biotage AB, Uppsala, Sweden). Sufficient amount of PCR-template of each SNP was incubated in a shaker (10 min) with streptavidin-coated sepharose beads (Amersham Pharmacia Biotech, Uppsala, Sweden) and prepared with 70% ethanol and denaturation buffer in a Vacuum Prep Workstation (pyrosequencing AB, Uppsala, Sweden) for transfer of the biotinylated templates into 55 μl of the corresponding 0.35 μmol/l sequencing primer. Sequencing took place after incubation for 2 min at 80° C.

### Classic Sequencing

To verify the correct genetic diagnosis as provided by the three assays, a total of 40 samples of wild-type (n=XX control samples), heterozygous (n=Xx control samples) and homozygous (n=XX control samples) genotype were conventionally sequenced (AGOWA, Berlin, Germany) and implemented as positive controls during pyrosequencing.

### Prediction of the Pain Protective 15-Position GCH1 Haplotype by Three SNPs

Based on the original haplotyping with 15 DNA positions, 78 heterozygous, 6 homozygous and 206 non-carriers of the pain protective haplotype were found, corresponding to an allelic frequency of this particular haplotype of 15.5% (binomial 95% confidence interval: 12.7%-18.7%).

Based on haplotype assignment including two DNA positions (dbSNP rs8007267 A and dbSNP rs3783641 T), 86 heterozygous and 6 homozygous carriers of the pain protective haplotype were predicted, corresponding to an allelic frequency of this particular haplotype of 16.8%. With eight false positive and no false negative assignments, the screening test sensitivity and specificity for haplotype #3 were 1 and 0.96, respectively. By haplotype assignment including three DNA positions (dbSNP rs8007267 A. dbSNP rs3783641 T, and dbSNP rs10483639 G), all the 78 heterozygous and 6 homozygous carriers of the pain protective haplotype were correctly predicted, corresponding of screening test sensitivity and specificity of 1. The number of homozygous and heterozygous subjects was in accordance with the Hardy-Weinberg equilibrium (χ²-test: p>0.05). Linkage between dbSNP rs8007267A, dbSNP rs3783641T0 and dbSNP rs10483639G in the 290 m samples (2) was indicated by D′=0.9, 0.81, and 0.88, respectively, and r²=0.78, 0.61, and 0.75, respectively.

The pain protective haplotype could be reliably assigned from the genetic information of the three SNPs, without in-silico haplotyping. That is, all homozygous carriers could he correctly predicted already on the basis of dbSNP rs8007267 G>A, i.e., all homozygous dbSNP rs8007267 AA carriers were homozygous for the pain protective haplotype, whereas dbSNP rs8007267 GG excluded the pain protective haplotype. With heterozygous dbSNP rs8007267 G>A, information from dbSNP rs3783641 A>T increased the specificity to detect the pain protective haplotype to 0.96, and additional information from dbSNP rs104836390G increased the specificity to 1. Similar effects have been achieved by analyzing combinations of rs8007267 G>A, rs3783641 A>T, rs8007201 T>G, rs4411417 A>G, and/or rs752688 G>A.

### CITED LITERATURE

1. Milstien S, Jaffe H, Kowlessur D, Bonner T I. Purification and cloning of the GTP cyclohydrolase I feedback regulatory protein, GFRP. J Biol. Chem. 1996 Aug. 16; 271(33):19743-51.
2. Tegeder I, Costigan M, Griffin R S, Abele A, Belfer I, Schmidt H, et al. The enzyme cofactor tetrahydrobiopterin modulates peripheral neuropathic pain. Nat Med 2006 (in press).
3. Cizkova D, Lukacova N, Marsala M, Marsala J. Neuropathic pain is associated with alterations of nitric oxide synthase immunoreactivity and catalytic activity in dorsal root ganglia and spinal dorsal horn. Brain Res Bull. 2002 June; 58(2):161-71.
4. Zeitz K P, Guy N, Malmberg A B, Dirajlal S, Martin W J, Sun L, Bonhaus D W, Stucky C L, Julius D, Basbaum A I. The 5-HT3 subtype of serotonin receptor contributes to nociceptive processing via a novel subset of myelinated and unmyelinated nociceptors. J. Neurosci. 2002 Feb. 1; 22(3):1010-9.
5. den Duunen J T, Antonarakis S E. Mutation nomenclature extensions and suggestions to describe complex mutations: a discussion. Hum Mutat 2000; 15(1):7-12.
6. Shi L, Ho J, Norling L A, Ray M, Xu Y. A real time quantitative PCR-based method for the detection and quantification of simian virus 40. Biologicals 1999; 27(3):241-52.
7. Stephens M, Smith N J, Donoelly P. A new statistical method for haplotype reconstruction from population data. Am J Hum Genet. 2001; 68(4).978-89.
8. Stephens M, Donnelly P. A comparison of bayesian methods for haplotype reconstruction from population genotype data. Am J Hum Genet. 2003; 73(5):1162-9.
9. Nyren P, Lundin A. Enzymatic method for continuous monitoring of inorganic pyrophosphate synthesis. Anal Biochem 1985; 151(2):504-9.
10. Ronaghi M, Karamohamed S, Pettersson B, Uhlen M, Nyren P. Real-time DNA sequencing using detection of pyrophosphate release. Anal Biochem. 1996 Nov. 1; 242(1):84-9.
11. Ichinose H, Ohye T, Takahashi E, Seki N, Hori T, Segawa M, Nomura Y, Endo K, Tanaka H, Tsuji S, et al. Hereditary progressive dystonia with marked diurnal fluctuation caused by mutations in the GTP cyclohydrolase I gene. Nat. Genet. 1994 November; 8(3):236-42.
12. Niederwieser A, Blau N, Wang M, Joller P, Atares M, Cardesa-Garcia J. GTP cyclohydrolase I deficiency, a new enzyme defect causing hyperphenylalmioemia with neopterin, hiopterin, dopamine, and serotonin deficiencies and muscular hypotonia. Eur J Pediatr 1984; 141(4):208-14.
13. Furukawa Y, Guttman M, Sparagana S P, Trugman J M, Hyland K, Wyatt P, et al. Dopa-responsive dystonia due to a large deletion in the GTP cyclohydrolase I gene. Ann Neurol 2000, 47(4):517-20.
14. Hagenah J, Saunders-Pullman R, Hedrich K, Kabakci K, Habermann K, Wiegers K, et al. High mutation rate in dopa-responsive dystonia: detection with comprehensive GCHI screening. Neurology 2005; 64(5):908-11.
15. Hoehe M R. Haplotypes and the systematic analysis of genetic variation in genes and genomes. Pharmacogenomics 2003; 4(5):547-70.
16. Hwu W L, Yeh H Y, Fang S W, Chiang H S, Chiou Y W, Lee Y M. Regulation of GTP cyclohydrolase I by alternative splicing in mononuclear cells. Biochem Biophys Res Commun. 2003 Jul. 11; 306(4):937-42.
17. Huang A, Zhang Y Y. Chen K, Hatakeyama K, Keeaney J F, Jr. Cytokine-stimulated GTP cyclohydrolase I expression in endothelial cells requires coordinated activation of nuclear factor-kappa B and Stat 1/Stat3. Circ Res 2005; 96(2):164.71
18. Al Sarraj J, Vinson C, Han J, Thiel G. Regulation of GTP cyclohydrolase I gene transcription by basic region leucine zipper transcription factors. J Cell Biochem 2005; 96(5):1003-20.
19. Comb M, Mermod N, Hyman S E, Pearlberg J, Ross M E, Goodman H M. Proteins bound at adjacent DNA elements act synergistically to regulate human proenkephalin cAMP inducible transcription. Embo J 1988; 7(12):3793-805.
20. Solovieva S, Leino-Arjas P, Saarela I, Luoma K, Raininko R, Riihimaki H. Possible association of interleukin I gene locus polymorphisms with low back pain. Pain 2004; 109(1-2):8-19.
21. Fillingim R B, Kaplan L, Staud R, Ness T J, Glover T L, Campbell C M, Mogil J S, Wallace M R. The A118G single nucleotide polymorphism of the mu-opioid receptor gene (OPRM1) is associated with pressure pain sensitivity in humans. J. Pain. 2005 March; 6(3):159-67.
22. Lotsch J, Stuck B. Hummel T. The human mu-opioid receptor gene polymorphism 118A>G decreases cortical activation in response to specific nociceptive stimulation. Behay. Neurosci. 2006 (in revision).

1-13. (canceled)

14. A high throughput automated screening system for determining a genetic predisposition or susceptibility for developing acute and/or chronic pain in a mammal, the system comprising:
a) a high throughput genotyping means, connected to
b) a computer processor and a computer-readable physical medium with software instructions encoded thereupon for a process, executable by said processor, said instructions comprising:
(i) instructions for receiving data regarding the genotype of at least one single nucleotide polymorphism (SNP) in a mammal at the genomic locus of the gene GCH1, wherein said at least one SNP is selected from the group consisting of the SNPs rs8007267 G>A, rs3783641 A>T, rs8007201 T>G, rs4411417 A>G, rs752688 G>A, and rs10483639 C>G;
(ii) instructions for comparing the genotype data received in (a) with reference genotype data regarding a protective haplotype for genetic predisposition or susceptibility for developing acute and/or chronic pain, wherein said comparing identifies whether said mammal carries a protective haplotype for genetic predisposition or susceptibility for developing acute and/or chronic pain; and
(iii) outputting the result of said comparison to a user interface.

15. The system of claim 14 wherein said reference genotype data comprises the GCH1 SNP protective haplotype rs8007267 G>A, rs3783641 A>T, and rs10483639 C>G, and wherein a mammal matching said SNP haplotype has a reduced genetic predisposition or susceptibility for developing acute and/or chronic pain relative to a mammal not matching said protective haplotype.

16. The system of claim 14 wherein said genotyping means is selected from means for allele-specific primer extension, allele-specific hybridization, allele-specific oligonucleotide ligation, and allele-specific cleavage of a flap probe.

17. The system of claim 14 wherein said instructions for outputting the result comprise instructions for outputting the result to a display.

18. A method for diagnosing a genetic predisposition or susceptibility for developing acute and/or chronic pain in a mammal, comprising detecting in a sample obtained from said mammal at least one single nucleotide polymorphism (SNP) in a nucleic acid or fragment thereof derived from the genomic locus of the gene GCH1, wherein said at least one SNP is selected from the group consisting of the SNPs rs8007267 G>A, rs3783641 A>T, rs8007201 T>G, rs4411417 A>G, rs752688 G>A, and rs10483639 C>G.

19. The method according to claim 18, wherein the diagnosis identifies individuals that are protected from said acute and/or chronic pain, in particular neuropathic pain.

20. The method according to claim 18, wherein said mammal is a human.

21. The method according to claim 18, wherein said nucleic acid is DNA, genomic DNA, RNA, cDNA, hnRNA and/or mRNA.

22. The method according to claim 18, wherein said detection is accomplished by sequencing, mini-sequencing, hybridization, restriction fragment analysis, oligonucleotide ligation assay or allele specific PCR.

23. The method according to claim 18, wherein a combination of SNPs provides for a detection with a sensitivity of about 0.80 or more, and a specificity of about 0.60 or more.

24. The method according to claim 18, wherein a combination of SNPs together with other statistically significant SNPs in the gene GCH1 is analyzed.

25. The method according to claim 18, wherein a combination of SNPs together with other statistically significant SNPs in a gene selected from the group of KCNS1, OPMR1, COMT, and PGHS2 is analyzed.

26. The method according to claim 18, further comprising an analysis of biopterin in whole blood and/or isolated leukocytes with and without a stimulation.

27. A method for producing an effective analgesic composition, the method comprising
a) performing a method according to claim 18,
b) determining a dosage of an analgesic substance for said mammal based, at least in part, on the result as obtained in step a), and
c) admixing said dosage with a pharmaceutically acceptable carrier and/or diluent.

28. A method of treating pain in a mammal, comprising
a) detecting in a sample obtained from said mammal at least one single nucleotide polymorphism (SNP) in a nucleic acid or fragment thereof derived from the genomic locus of the gene GCH1, wherein said at least one SNP is selected from the group consisting of the SNPs rs8007267 G>A, rs3783641 A>T, rs8007201 T>G, rs4411417 A>G, rs752688 G>A, and rs10483639 C>G; and
b) providing an analgesic substance to said mammal in a dosage adjusted on the basis of the result as obtained in step (a).

29. The method according to claim 28, wherein the analgesic substance administered is an effective analgesic composition produced by the method of claim 27.

30. A kit, comprising at least one probe and/or set of primers for detecting at least one of the SNPs of the gene GCH1, selected from the group consisting of the SNPs rs8007267 G>A, rs3783641 A>T, rs8007201 T>G, rs4411417 A>G, rs752688 G>A, and rs10483639 C>G.
